(19) United States
(12) Patent Application Publication
(10) Pub. No.: US 2006/0211931 A1
(43) Pub. Date: Sep. 21, 2006

(54) Noninvasive analyzer sample probe interface method and apparatus

(76) Inventors: Thomas B. Blank, Gilbert, AZ (US); Mutua Mattu, Chandler, AZ (US); James R. Henderson, Phoenix, AZ (US); Kevin H. Hazen, Gilbert, AZ (US); Roxanne Abul-Haj, Mesa, AZ (US)

Correspondence Address: GLENN PATENT GROUP, 3475 EDISON WAY, SUITE L, MENLO PARK, CA, 94025, US

(21) Appl. No.: 11/359,700

(22) Filed: Feb. 21, 2006

Related U.S. Application Data
(63) Continuation-in-part of application No. 11/008,001, filed on Dec. 8, 2004.
Application No. 11/008,001 is a continuation-in-part of application No. 09/563,782, filed on May 2, 2000, now Pat. No. 6,415,167.
Application No. 11/008,001 is a continuation-in-part of application No. 10/170,921, filed on Jun. 12, 2002.
Application No. 10/170,921 is a continuation-in-part of application No. 09/563,782, filed on May 2, 2000, now Pat. No. 6,415,167.
(63) Continuation-in-part of application No. 10/472,856, filed on Sep. 18, 2003.
(63) Continuation-in-part of application No. 11/117,104, filed on Apr. 27, 2005.
(60) Provisional application No. 60/362,899, filed on Mar. 8, 2002.
(60) Provisional application No. 60/655,923, filed on Feb. 23, 2005.
(60) Provisional application No. 60/566,568, filed on Apr. 28, 2004.
(60) Provisional application No. 60/656,727, filed on Feb. 25, 2005.
(60) Provisional application No. 60/658,708, filed on Mar. 3, 2005.
(60) Provisional application No. 60/761,486, filed on Jan. 23, 2006.

(51) Int. Cl. A61B 5/00

(52) U.S. Cl. 600/344; 600/310; 600/316

(57) ABSTRACT

The invention provides an adaptive mount for use in coupling a noninvasive analyte property analyzer to a living tissue sample site. The adaptive mount increases precision and accuracy of sampling by relieving stress and strain on a sample prior to and/or during sampling, which results in noninvasive analyte property estimations with corresponding performance enhancement.

### CROSS REFERENCE TO RELATED APPLICATIONS

This application is a continuation in part of:
U.S. patent application Ser. No. 11/008,001 filed Dec. 8, 2004 (attorney docket no. IMET0045CIP2), which is a continuation in part of U.S. patent application Ser. No. 09/563,782 and U.S. patent application Ser. No. 10/170,921, which is a continuation in part of U.S. patent application Ser. No. 09/563,782;
U.S. patent application Ser. No. 10/472,856 filed Mar. 7, 2003 (attorney docket no. SENS0011), which claims:
   priority to PCT application no. PCT/US03/07065;
   benefit of U.S. provisional patent application no. 60/362,899; and
   benefit of U.S. provisional patent application no. 60/362,885; and
U.S. patent application Ser. No. 11/117,104, filed Apr. 27, 2005 (attorney docket no. SENS0050), which claims benefit of U.S. provisional application no. 60/566,568, filed Apr. 28, 2004;
   and claims benefit of:
U.S. provisional patent application no. 60/656,727 filed Feb. 25, 2005 (attorney docket no. SENS0059PR);
U.S. provisional patent application no. 60/658,708 filed Mar. 3, 2005 (attorney docket no. SENS0059PR2); and
U.S. provisional patent application no. 60/761,486 filed Jan. 23, 2006 (attorney docket no. SENS0065PR);
   all of which are incorporated herein in their entirety by this reference thereto.

### BACKGROUND OF THE INVENTION

1. Field of the Invention

The invention relates to noninvasive sampling. More particularly, the invention relates to a sample probe interface method and apparatus for use in conjunction with a spectroscopy based noninvasive analyzer. More particularly, the invention relates a mount and placement of a mount for use with a noninvasive analyzer in a manner that facilitates improved accuracy and precision of subsequent optical measurements and analyte property determinations associated with the optical measurements.

2. Description of Related Art

Spectroscopy based noninvasive analyzers deliver external energy in the form of light to a specific sample site, region, or volume of the human body where the photons interact with a tissue sample, thus probing chemical and physical features. Portions of the incident photons are specularly reflected, diffusely reflected, scattered, or transmitted out of the body where they are detected. Based upon knowledge of the incident photons and detected photons, the chemical and/or structural basis of the sampled site is deduced. A distinct advantage of a noninvasive analyzer is the analysis of chemical and structural constituents in the body without the generation of a biohazard in a pain-free manner with limited consumables. Additionally, noninvasive analyzers allow multiple analytes or structural features to be determined at one time. Common examples of noninvasive analyzers are magnetic resonance imaging (MRI's), X-rays, pulse oximeters, and noninvasive glucose concentration analyzers. With the exception of X-rays, these determinations are performed with relatively harmless wavelengths of radiation. Examples herein focus on noninvasive glucose concentration determination, but the principles apply to other noninvasive measurements and/or determination of additional blood or tissue analyte properties.

Diabetes

Diabetes is a chronic disease that results in abnormal production and use of insulin, a hormone that facilitates glucose uptake into cells. While a precise cause of diabetes is unknown, genetic factors, environmental factors, and obesity play roles. Diabetics have increased risk in three broad categories: cardiovascular heart disease, retinopathy, and neuropathy. Diabetics often have one or more of the following complications: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance and hyperinsulinemia, which is also known as hypoglycemia.

Diabetes Prevalence and Trends

The prevalence of individuals with diabetes is increasing with time. The World Health Organization (WHO) estimates that diabetes currently afflicts 154 million people worldwide. There are 54 million people with diabetes living in developed countries. The WHO estimates that the number of people with diabetes will grow to 300 million by the year 2025. In the United States, 18.2 million people or 6.9 percent of the population are estimated to have diabetes, which is an increase of 40% between 1992 and 2002. This corresponds to approximately eight hundred thousand new cases every year in America. The estimated total cost to the United States economy alone exceeds $90 billion per year. Diabetes Statistics, National Institutes of Health, Publication No. 98-3926, Bethesda, Md. (November, 1997); JAMA, vol. 290, pp. 1884-1890 (2003).

Long-term clinical studies demonstrate that the onset of diabetes related complications is significantly reduced through proper control of blood glucose concentrations [The Diabetes Control and Complications Trial Research Group, The effect of intensive treatment of diabetes on the development and progression of long-term complications in insulin-dependent diabetes mellitus, N. Eng. J. of Med., 329:977-86 (1993); U.K. Prospective Diabetes Study (UKPDS) Group, Intensive blood-glucose control with sulphonylureas or insulin compared with conventional treatment and risk of complications in patients with type 2 diabetes, Lancet, 352:837-853 (1998); and Y. Ohkubo, H. Kishikawa, E. Araki, T. Miyata, S. Isami, S. Motoyoshi, Y. Kojima, N. Furuyoshi, M. Shichizi, Intensive insulin therapy prevents the progression of diabetic microvascular complications in Japanese patients with non-insulin-dependent diabetes mellitus: a randomized prospective 6-year study, Diabetes Res. Clin. Pract., 28:103-117 (1995)].

A vital element of diabetes management is the self-monitoring of blood glucose concentration by diabetics in the home environment. However, current monitoring techniques discourage regular use due to the inconvenient and painful nature of drawing blood or interstitial fluid through the skin prior to analysis, The Diabetes Control and Complication Trial Research Group, supra. As a result, noninvasive measurement of glucose concentration is identified as a beneficial development for the management of diabetes. Implantable glucose analyzers coupled to an insulin delivery system providing an artificial pancreas are also being pursued.

Noninvasive Glucose Concentration Determination

There exist a number of noninvasive approaches for glucose concentration determination in tissue or blood. These approaches vary widely but have at least two common steps. First, an apparatus is used to acquire a photometric signal from the body. Second, an algorithm is used to convert this signal into a glucose concentration determination.

One type of noninvasive glucose concentration analyzer is a system performing glucose concentration estimations from spectra. Typically, a noninvasive apparatus uses some form of spectroscopy to acquire a signal, such as a spectrum, from the body. A particular range for noninvasive glucose concentration determination in diffuse reflectance mode is in the near-infrared from approximately 1100 to 2500 nm or one or more ranges therein. These techniques are distinct from the traditional invasive and alternative invasive techniques in that the interrogated sample is a portion of the human body in-situ, not a biological sample acquired from the human body.

There are a number of reports on noninvasive glucose technologies. Some of these relate to general instrumentation configurations required for noninvasive glucose concentration determination while others refer to sampling technologies. Those related to the present invention are briefly reviewed here:

General Instrumentation

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration determination analyzer that uses data pretreatment in conjunction with a multivariate analysis to determine blood glucose concentrations.

P. Rolfe, Investigating substances in a patient's bloodstream, United Kingdom patent application ser. no. 2,033,575 (August 24, 1979) describes an apparatus for directing light into the body, detecting attenuated backscattered light, and uses the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentrations from selected near-infrared wavelength bands. Wavelengths include 1560 to 1590, 1750 to 1780, 2085 to 2115, and 2255 to 2285 nm with at least one additional reference signal from 1000 to 2700 nm.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose, using infrared spectroscopy in conjunction with a multivariate model. The multivariate model is constructed from a plurality of known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-infrared. A plurality of distinct nonoverlapping regions of wavelengths are incident upon a sample surface, diffusely reflected radiation is collected, and the analyte concentration is determined via chemometric techniques.

Positioning

E. Ashibe, Measuring condition setting jig, measuring condition setting method and biological measuring system, U.S. Pat. No. 6,381,489, Apr. 30, 2002 describes a measurement condition setting fixture secured to a measurement site, such as a living body, prior to measurement. At time of measurement, a light irradiating section and light receiving section of a measuring optical system are attached to the setting fixture to attach the measurement site to the optical system.

J. Röper, D. Böcker, System and method for the determination of tissue properties, U.S. Pat. No. 5,879,373 (Mar. 9, 1999) describe a device for reproducibly attaching a measuring device to a tissue surface.

T. Blank, G. Acosta, M. Mattu, S. Monfre, Fiber optic probe guide placement guide, U.S. Pat. No. 6,415,167 (Jul. 2, 2002) describe a coupling fluid and the use of a guide in conjunction with a noninvasive glucose concentration analyzer in order to increase precision of the location of the sampled tissue site resulting in increased accuracy and precision in noninvasive glucose concentration estimations.

T. Blank, G. Acosta, M. Mattu, M. Makarewicz, S. Monfre, A. Lorenz, T. Ruchti, Optical sampling interface system for in-vivo measurement of tissue, world patent publication no. WO 2003/105664 describe an optical sampling interface system that includes an

. optical probe placement guide, a means for stabilizing the sampled tissue, and an optical coupler for repeatably sampling a tissue measurement site in-vivo.

J. Griffith, P. Cooper, T. Barker, Method and apparatus for non-invasive blood glucose sensing, U.S. Pat. No. 6,088,605 (Jul. 11, 2000) describe an analyzer with a patient forearm interface in which the forearm of the patient is moved in an incremental manner along the longitudinal axis of the patient's forearm. Spectra collected at incremental distances are averaged to take into account variations in the biological components of the skin. Between measurements rollers are used to raise the arm, move the arm relative to the apparatus, and lower the arm by disengaging a solenoid causing the skin lifting mechanism to lower the arm into a new contact position with the sensor head.

Temperature

K. Hazen, Glucose Determination in Biological Matrices Using Near-Infrared Spectroscopy, doctoral dissertation, University of Iowa (1995) describes the adverse effect of temperature on near-infrared based glucose concentration estimations. Physiological constituents have near-infrared absorbance spectra that are sensitive, in terms of magnitude and location, to localized temperature and the sensitivity impacts noninvasive glucose concentration determination.

Pressure

E. Chan, B. Sorg, D. Protsenko, M. O′Neil, M. Motamedi, A. Welch, Effects of compression on soft tissue optical properties, IEEE Journal of Selected Topics in Quantum Electronics, Vol. 2, no. 4, pp. 943-950 (1996) describe the effect of pressure on absorption and reduced scattering coefficients from 400 to 1800 nm. Most specimens show an increase in the scattering coefficient with compression.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe in a first embodiment a noninvasive glucose concentration estimation apparatus for either varying the pressure applied to a sample site or maintaining a constant pressure on a sample site in a controlled and reproducible manner by moving a sample probe along the z-axis perpendicular to the sample site surface. In an additional described embodiment, the arm sample site platform is moved along the z-axis that is perpendicular to the plane defined by the sample surface by raising or lowering the sample holder platform relative to the analyzer probe tip. The '012 patent further teaches proper contact to be the moment specularly reflected light is about zero at the water bands at 1950 and 2500 nm.

M. Makarewicz, M. Mattu, T. Blank, G. Acosta, E. Handy, W. Hay, T. Stippick, B. Richie, Method and apparatus for minimizing spectral interference due to within and between sample variations during in-situ spectral sampling of tissue, U.S. patent application Ser. No. 09/954,856 (filed Sep. 17, 2001) describe a temperature and pressure controlled sample interface. The means of pressure control are a set of supports for the sample that control the natural position of the sample probe relative to the sample.

To date, however, accurate and precise noninvasive glucose concentration estimations have not been generated in a reproducible fashion, largely due to the changing nature of the sampled biological matrix itself. Particularly, skin moves, stretches, expands and contracts, and/or undergoes torque before, between, and/or during sampling. This results in structural changes to the sample site and changes in physical properties that contribute error to noninvasive analyte property estimations. A need exists for a noninvasive analyzer sample interface that adapts to the changing structure of skin.

### SUMMARY OF THE INVENTION

The invention provides an adaptive mount for use in coupling a noninvasive analyte property analyzer to a living tissue sample site. The adaptive mount increases precision and accuracy of sampling by relieving stress and strain on a sample prior to and/or during sampling, which results in noninvasive analyte property estimations with corresponding performance enhancement.

### BRIEF DESCRIPTION OF THE DRAWINGS

FIG. 1 is a perspective view of a noninvasive glucose concentration analyzer according to the invention;

FIG. 2 provides a block diagram of an analyzer according to the invention;

FIG. 3 is a schematic of a two-piece guide interfacing with tissue;

FIG. 4 provides a schematic of an adaptive mount adapting to tissue state change according to the invention;

FIG. 5 is a perspective representation of an adaptive mount according to the invention;

FIG. 6 provides a perspective representation of an adaptive mount with side and end views according to the invention;

FIG. 7 illustrates a guide and a mount according to the invention; and

FIG. 8 presents a controller driving an actuator that moves a sample probe relative to a sample according to the invention.

### DETAILED DESCRIPTION OF THE INVENTION

The invention provides a solution that adapts to the changing structure of skin by relaxing constraints that a guide or jig imposes upon the sample site, such as forcing a fixed location to be sampled with each measurement. An adaptive mount, which relieves strain on the sample site between and during sampling, is used to overcome changes in the sample site. Use of a mount constrains position of sampling to a lesser degree than with a guide resulting in sampling variations result. It has been determined that standard chemometric approaches adequately compensate for small variations in sample position more effectively than chemometric approaches compensate for spectral variation due to stress on the sample. Therefore, an adaptive sample probe mount that reduces stress and strain results in improved precision and accuracy of noninvasive analyte property estimation.

An adaptive mount results in:
increased precision and accuracy of noninvasive sampling; and
a means of assuring that the similar tissue sample volumes are repeatably sampled by minimizing sampling errors due to mechanical tissue distortion and probe placement.

An adaptive mount is presented that increases precision and accuracy of noninvasive sampling, which results in increased sensitivity, precision, and accuracy of subsequent analyte property estimation derived from the sampling. The adaptive mount is placed onto the skin of a person. Between uses, opposing ends of the adaptive mount move relative to each other as the skin tissue changes state. During use, the adaptive mount minimizes skin deformation during placement of a sample probe of an analyzer or during placement of a plug. In a first embodiment of the invention, the adaptive mount samples a dynamic x-, y-position at or about a central sample site. In another embodiment of the invention, the adaptive mount is deformable, which distributes applied forces during sample about the sample site. Detailed descriptions of these embodiments and the interaction of the dynamic mount with a noninvasive analyzer are provided, infra.

In spectroscopic analysis of living tissue, it is often necessary to sample optically at or near a given tissue volume repeatedly through the use of an optical probe; for example while developing a noninvasive calibration for measuring one or more tissue analytes, and subsequently, when taking measurements for the actual analyte measurement. Sampling errors are often introduced into these measurements because of the difficulty of repeatedly placing the optical probe at the precise location used in preceding measurements, and due to repeatably producing the same nominal degree of tissue distortion and displacement with each sample acquisition. With each small variation in the location of the probe, or variations in the amount of pressure resulting from the repeated probe contact events, a slightly different tissue volume is sampled, thereby introducing sampling errors into the measurements. The invention provides an optical sampling interface system that eliminates or minimizes factors that account for sampling error.

Tissue Strain

Strain is the elongation of material under load. Stress is the increased internal energy inherent in a material under strain. For an elongated material to have strain there must be resistance to stretching. For example, an elongated spring has strain characterized by percent elongation, such as percent increase in length. The stress is the potential energy of the elongated spring.

Skin contains constituents, such as collagen, that have spring-like properties. That is, elongation causes an increase in potential energy of the skin. Strain induced stress changes optical properties of skin, such as absorbance and scattering. Therefore, it is undesirable to make optical spectroscopy measurements on skin under various stress states. Stressed skin also causes fluid movements that are not reversible on a short timescale. The most precise optical measurements are therefore conducted on skin in the natural strain state, such as minimally stretched skin. Skin is stretched or elongated by applying loads to skin along any of the x-, y-, and z-axes, described infra.

An adaptive probe mount system that compensates for geometric changes in skin structure provides the best measurement potential as optical homogeneity has low variation over short x-, y-distances, such as less than one millimeter. When using a sufficiently large optical aperture at the probe/skin interface, the homogeneity variation over small x-, y- distances are either negligible or compensable with chemometric techniques. The dynamic probe mount minimizes skin stress and corresponding optical scattering changes and fluid movements in skin caused by the measurement perturbation.

Analyzers are typically used to determine an analyte property, such as concentration. However, when complex models or soft models are used, analyzers typically estimate an analyte property, such as concentration. Herein, the term estimation is used interchangeably with determination.

Analyzer

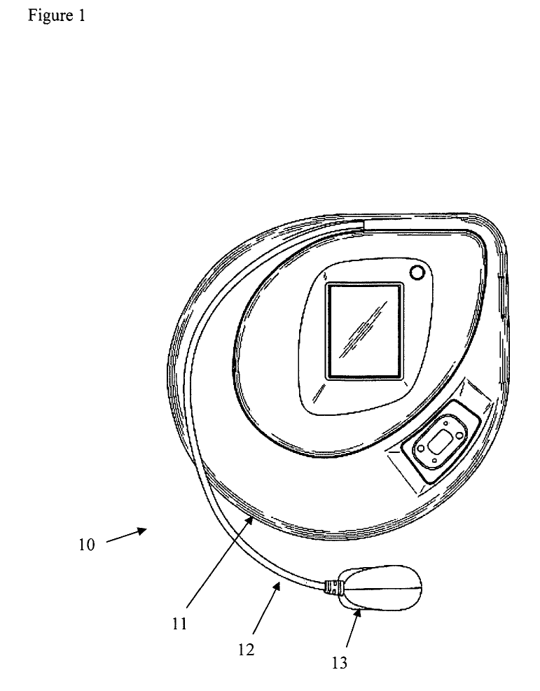

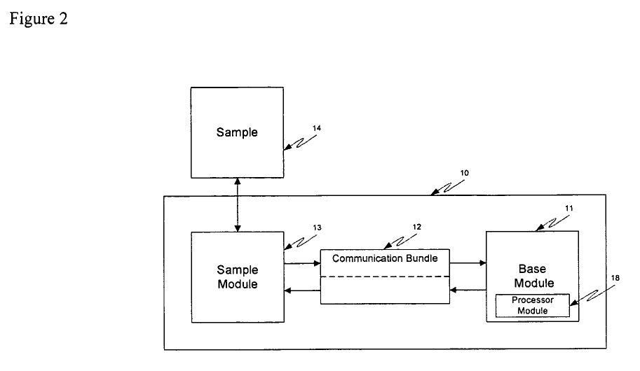

An analyte estimation and/or concentration tracking system is used, such as a glucose concentration tracking system. Herein, a noninvasive analyzer, such as a glucose concentration analyzer, comprises at least a source, a sample interface, at least one detector, and an associated algorithm. Referring now to FIG. 1, an example of a glucose concentration analyzer is presented. In FIG. 1, an analyzer 10 is separated into elements including a base module 11, a communication bundle 12, and a sample module 13. The advantages of separate units are hereinafter described. The sample module, also referred to as a sampling module, interfaces with a tissue sample and at the same or different times with one or more reference materials. Herein, the combined base module 11, communication bundle 12, sample module 13, and algorithm are referred to as a spectrometer and/or analyzer 10. Referring now to FIG. 2, a block diagram, including a processor module 18, of an analyzer is provided.

Traditionally, the base module and sample module are in a single housing. For example, the components of a noninvasive glucose analyzer are included in a single unit, such as a professional use analyzer, a stand-alone analyzer, or a handheld analyzer. In the example illustrated in FIG. 1, the base module and sample module are in separate housings. Providing separate housings for the sample module and base module has multiple benefits, such as easing thermal, size, and weight management by allowing the sources of these features to be separated into multiple housings. For example, the sample module is allowed to be smaller and weigh less without the bulk of the base module. This eases handling by the user and results in decreased physical impact on the sample site during sampling of tissue by the sample module, infra. In a further example, heat from a source in one housing is separated from a detector in a second housing allowing for ease in cooling the detectors, thereby resulting in lower detector noise. The sample module, base module, and communication bundle are further described, infra.

Sample Module

A sample module includes a sensor head assembly or sample probe that provides an interface between the analyzer, such as a glucose concentration tracking system, and the patient or sample site. The tip of the sample probe of the sample module is brought into contact with the tissue sample. Optionally, the tip of the sample probe is interfaced to an adaptive mount, such as an arm-mounted adaptive probe mount, to conduct data collection and is typically removed when the process is complete. Optional mount accessories include an occlusion plug for hydrating and/or protecting the sample site surface and means for photo-stimulation to enhance circulation. The occlusion plug is optionally used when the sensor head is not inserted in the mount. In one example, the following components are included in the sample module sensor head assembly: a light source, a single fiber optic, and coupling fluid. In a second example, the sample module includes at least one light directing optic and means for interfacing to an adaptive mount.

Preferably, the sample module is in a separate housing from the base module. Alternatively, the sample module is integrated into a single unit with the base module, such as in a handheld or desktop analyzer. In this alternative embodiment, the communication bundle is wireless or is integrated into the analyzer.

Communication Bundle

A communication bundle is a multi-purpose bundle. The multi-purpose bundle is a flexible sheath that includes at least one of:
electrical wires to supply operating power to a lamp in the sample module;
thermistor wires;
one or more fiber-optics, which direct diffusely reflected near-infrared light to a spectrograph;
a tube, used to transport optical coupling fluid from the base unit, through the sensor head, and onto the measurement site;
a tension member to remove loads on the wiring and fiber-optic strand from pulls;
an optical mixing tube; and
photo sensor wires.

In alternative embodiments of the invention, the communication bundle is absent and signals are transmitted and received between the base module and sample module using wireless technology.

Base module

A signal is communicated from the sample module to a base module. Preferably, a portion of the diffusely reflected light from the site is collected and transferred via at least one fiber-optic, free space optics, digitally after detection, or via an optical pathway to the base module. Preferably, the base module contains a wavelength separation device, such as a spectrograph, grating, or a time resolved or spatially resolved system for wavelength separation. The spectrograph separates the spectral components of the diffusely reflected light, which are then directed to one or more detectors, such as a photo-diode array (PDA). In the instance that a PDA is used, the PDA converts the sampled light into a corresponding analog electrical signal, which is then preferably conditioned by analog front-end circuitry. The analog electrical signals are typically converted into their digital equivalents by the analog circuitry. The digital data are then sent to the digital circuitry where they are checked for validity, processed, and stored in non-volatile memory. Optionally, the processed results are recalled when the session is complete and after additional processing the individual analyte property is available for display or transfer to a digital device, such as a personal computer. The base module also, preferably, includes a central processing unit or equivalent for processors, memory, storage media for storing data, a model, a multivariate model, and/or analysis routines, such as those employing a model or net analyte signal.

Any of the embodiments described herein are operable in a home environment, public facility, or in a medical environment, such as an emergency room, critical care facility, intensive care unit, hospital room, or medical professional patient treatment area. For example, the split analyzer is operable in a critical care facility where the sample module is positioned in proximate contact with a subject or patient during use and where the base module is positioned on a support surface, such as a rack, medical instrumentation rack, table, or wall mount. Optical components, such as a source, backreflector, guiding optics, lenses, filters, mirrors, a wavelength separation device, and at least one detector are optionally positioned in the base module and/or sample module.

Adaptive Mount

A system is described herein that provides superior sampling precision of targeted tissue through the use of an adaptive mount or an adaptive sample probe mount that is removably attached about the tissue site. A key characteristic of the adaptive mount is achievement of highly repeatable sampling by limiting stress and strain on and about the median targeted tissue measurement site. To achieve this, the mount adapts to physical changes in the sample.

An additional benefit of the adaptive mount is that it optionally provides a means for locally registering the location of the targeted tissue volume with respect to the optical probe and/or tip of a sample module, such that a narrow range of tissue volumes are sampled by the optical system. Local registration refers to controlling the position of the optical probe relative to a target location on the tissue. The adaptive mount allows flexibility in terms of the exact position of the tissue that is sampled. This allows the sample to undergo stress, expand, contract, and/or twist and the mount adapts to the new state of the sample by mounting a sample probe to a slightly new position in terms of x-position and y-position, described infra. Means for registering the mount and the optical probe are optionally mechanical, optical, electrical, and/or magnetic.

A number of embodiments of the invention are described, infra. Additional embodiments are envisioned that are permutations and combinations of the adaptive mount components and/or accessories of the various described embodiments.

Coordinate system

Herein, an x, y, and z coordinate system relative to a given body part is defined. An x,y,z coordinate system is used to define the sample site, movement of objects about the sample site, changes in the sample site, and physical interactions with the sample site. The x-axis is defined along the length of a body part and the y-axis is defined across the body part. As an illustrative example using a sample site on the forearm, the x-axis runs between the elbow and the wrist and the y-axis runs across the axis of the forearm. Similarly, for a sample site on a digit of the hand, the x-axis runs between the base and tip of the digit and the y-axis runs across the digit. Together, the x,y plane tangentially touches the skin surface, such as at a sample site. The z-axis is defined as orthogonal to the plane defined by the x- and y-axis. For example, a sample site on the forearm is defined by an x,y plane tangential to the sample site. An object, such as a sample probe, moving along an axis perpendicular to the x,y plane is moving along the z-axis. Rotation of an object about one or a combination of axis is further used to define the orientation of an object, such as a sample probe, relative to the sample site. Tilt refers to an off z-axis alignment of the longitudinal orientation of the sample probe where the longitudinal axis extends from the sample probe tip interfacing with a sample site to the opposite end of the sample probe.

Guide / Mount

A guide is distinguished from an adaptive mount herein. A two-piece guide positions an external object, such as a sample probe, to the same x-, y-, z-position of a tissue sample. As the state of the skin changes, the guide forces the skin back to its original position so that the external object couples to the same x-, y-, z-position of the tissue sample. An adaptive mount positions an external object, such as a sample probe, to varying positions of a tissue sample. As the state of the skin changes, the adaptive mount moves with the tissue. The adaptive mount then adjusts the position of the external object relative to the tissue sample site. In this manner, the skin undergoes minimal stress because the skin is not deformed to force the exact same position of the tissue to be sampled with each measurement. Examples of a guide and an adaptive mount are provided, infra.

Two-Piece Guide

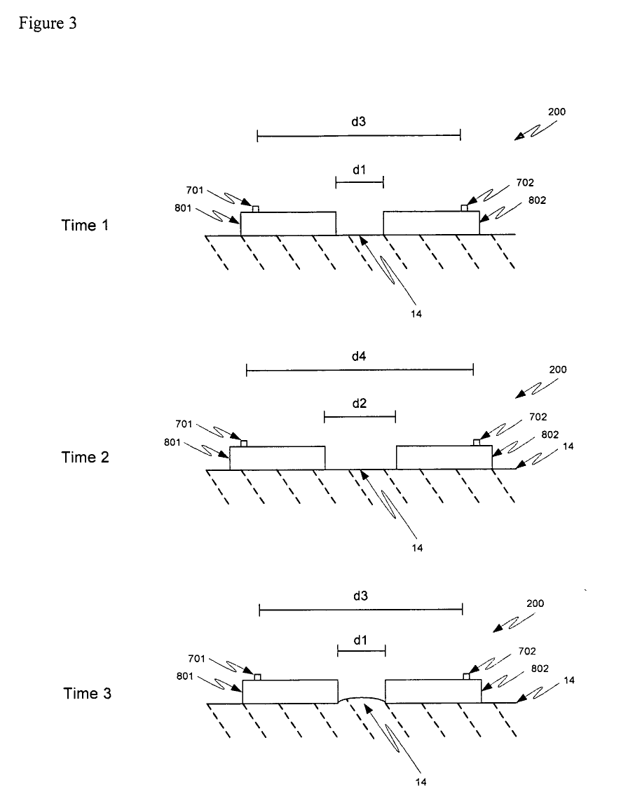

Referring now to FIG. 3, a two-piece guide 200 is presented that has a first alignment piece 801 and a second alignment piece 802. The first alignment piece 801 has a first registration piece 701 and the second alignment piece 802 has a second registration piece 702. Combined, one or more registration pieces, such as two registration pieces 701, 702, on the guide 200 preferably control the x-, y-, and z-position, as well as the rotational alignment of a corresponding sample probe. In the example presented in FIG. 3, the first alignment piece and second alignment piece are initially positioned on a tissue sample, at time 1, with a distance, d1, between the alignment pieces 801, 802. Initially, there is a distance d3 between the registration pieces 701, 702. At time 2, the state of the tissue 14 has changed resulting in an elongation of the tissue. This elongation results in the distance between the alignment pieces 801, 802 expanding to distance d2. The corresponding distance between the registration pieces 701, 702 has similarly expanded to distance d4. At time 3, the sample probe is aligned versus the two registration pieces 701, 702. The sample probe is designed with a fixed distance d3 between the alignment positions that correspond to the two registration pieces 701, 702 of the guide 200 as originally placed at time 1. For the lock and key mechanism of the sample probe and guide 200 to fit, the alignment pieces 701, 702 of the guide 200 are forced together to provide a spacing between the alignment pieces 801, 802 of distance d1. Because the alignment pieces are attached to the tissue 14, the tissue 14 deforms. The deformation or stress on the tissue 14 results in strain on the tissue 14 that is observed optically, supra. Similarly, if the tissue 14 contracts or twists between measurements, the guide 200 forces the tissue back into a state with distance d3 between the registration pieces 701, 702 at the time of sampling. This results in stress on the tissue 14 and corresponding strain on the tissue 14 that is observed through optical sampling of the tissue site 14. Typically, sampling change due to stress is detrimental to noninvasive analyte property determination. Additional embodiments of guides and mounts are described in U.S. patent application Ser. No. 11/008,001, which is incorporated herein in its entirety by this reference thereto.

A guide 200 controls a plurality of the rotation and x-, y-, and z-position of the sample probe relative to the tissue 14. In a two-piece guide, many combinations exist where the first registration piece 701 and second registration piece 702 each control one or more of the x-position, y-position, z-position, and rotational alignment of the sample probe. A commonality of a guide is that as the tissue changes, the tissue is deformed upon placement of a sample probe on the guide.

Adaptive Mount

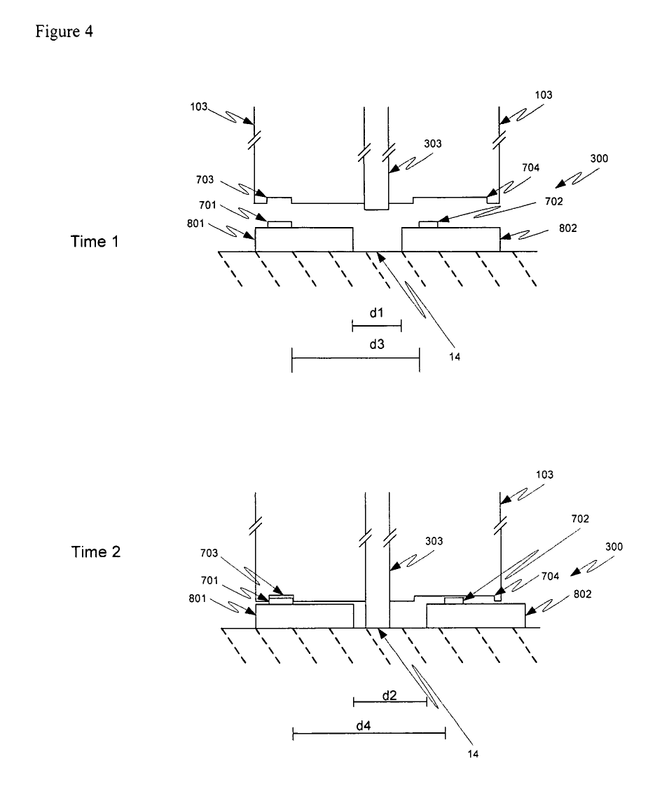

One embodiment of the invention includes an adaptive mount that is used to position a sample probe relative to a sample site. In this embodiment, at least one axis of the sample probe is allowed to float relative to a fixed x,y-point that defines a given sample site. Referring now to FIG. 4, an example of an adaptive mount with freedom of motion along the x-axis is presented at two moments in time. At time 1, the tissue 14 has a distance, d1, between a first alignment piece 801 and a second alignment piece 802. The two alignment pieces 801, 802 have corresponding means for registration 701, 702.

At time 1, the two registration pieces 701, 702 have a distance, d3, between them. In this case, the registration pieces protrude from the alignment pieces. Additional embodiments of registration pieces and alignment pieces are described, infra and in U.S. patent application Ser. No. 11/008,001. A portion of a sample module 13 is represented near the tissue 14. Registration pieces 703, 704 correspond to the registration pieces on the mount 701, 702, respectively. In this case, registration piece 703 acts as one-half of a lock and key element corresponding to the second half of a lock and key element 701. A sample probe 303 is situated at a given x-, y-position relative to the tissue 14.

At time 2, the tissue 14 has changed state. In the state pictured, the tissue has elongated, causing the distance between the first and second alignment pieces 801, 802 to expand in distance from d1 to d2. The corresponding distance between the first and second registration pieces 701, 702 has similarly expanded in distance from d3 to d4. If a guide with a fixed distance d3 between registration pieces is coupled to the tissue illustrated at time 2, then the tissue 14 deforms, such that the guide 200 couples to the sample module 13. In the current embodiment, the sample module 13 includes one registration piece 703 that couples with a corresponding registration piece 801 on the mount 200. A second registration piece 704 on the sample module 13 has freedom of movement in at least one-dimension relative to the alignment piece 802 and/or registration piece 702. The tip of the sample probe 303 mounts to a slightly different x-, y-position of the tissue 14 as the tissue state changes in a manner that effects the tissue size, shape, and or torque. This results in at least a portion of the sample module 13 and/or sample probe 303 to mount on the mount 300 via one or more alignment pieces and/or one or more registration pieces with minimal deformation or strain on the tissue 14. The mounting of the sample probe 303 to the mount 300 with minimal strain results in noninvasive spectra with fewer spectral interferences and hence corresponding analyte property estimation is more precise and accurate. Optionally, the sample probe 303 is movable along the z-axis, so that the tip of the sample probe results in minimal stress on the sample tissue volume. In the pictured instance, the sample probe is shown as extended to the tissue 14 at time 2. A movable z-axis sample probe is described, infra.

Similarly, the variable placement of the sample probe relative to the tissue is performed along the y-axis or through a combination of x- and y-axis. For example, the alignment piece 802 optionally contains means, such as groove along the y-axis for y-axis freedom of movement or a slide, such as a planar surface, for x- and y-axis freedom of movement.

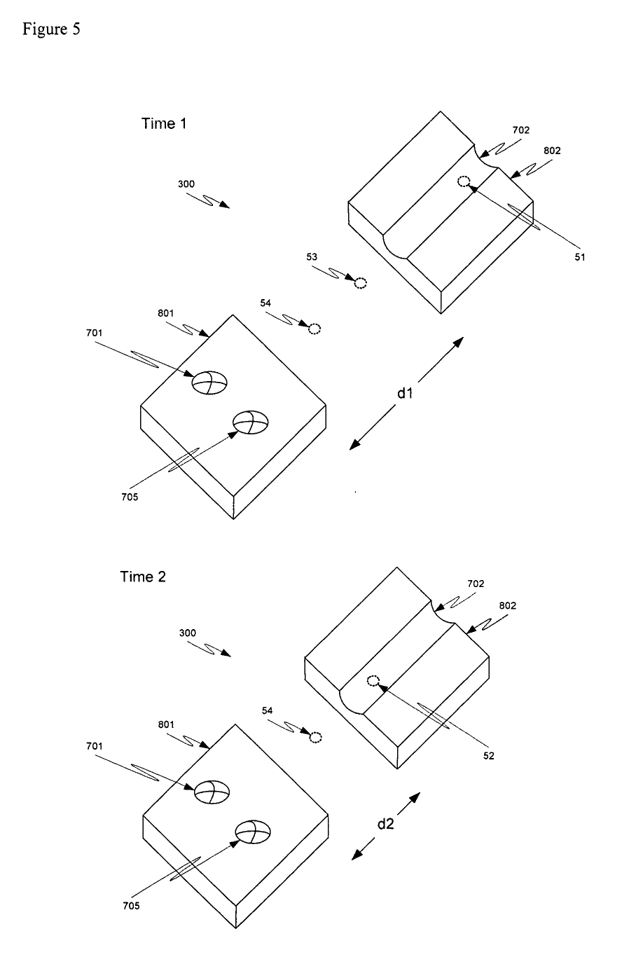

Referring now to FIG. 5, an additional example of an adaptive mount is presented. In this example, a perspective view is presented at time 1 of a first and second alignment piece 801, 802 placed about a tissue sample site that are separated by a distance d1. Two rounded registration pieces 701, 705 extend from the first registration piece 801 and a third registration piece 702 is a trough along the x-axis of the second alignment piece 802. A sample probe, not pictured in this view for clarity, is mounted by the three registration pieces 701, 702, and 705, such that the center of the optical sampling is about an area 53. At time two, the skin has contracted and the two registration pieces are now separated by a distance d2. The adaptive sample probe mounted on alignment pieces 701, 705, and 702 now samples an area 54. In this example, the two registration pieces 701, 705 prevent rotation of the sample probe. A separate registration piece, such as registration piece 702 allows movement of the sample probe along the x-axis, thereby allowing the sample probe position to adapt via the mount to the change in tissue.

The system allows the sample probe to be placed in terms of rotation, x-position, and y-position relative to the tissue with minimal stress applied to the tissue as the sample probe changes location relative to the skin as opposed to forcing the skin to undergo strain through adaptation to a fixed sample probe guide alignment. In additional instances, the two sampled areas 53, 54 overlay, overlap, or are separated. As described, infra, this example allows twisting or torque of the tissue sample. In an additional embodiment, the sample probe is moved dynamically along the z-axis, described infra. In still another embodiment, the alignment piece 802 has registration means that register in the y-axis or in a combination of axes. For example, the trough 702 runs along the y-axis to allow y-axis freedom of movement. In yet another embodiment of the invention, freedom of movement of the sample probe is provided in two-dimensions, such as with two troughs aligned normal to each other.

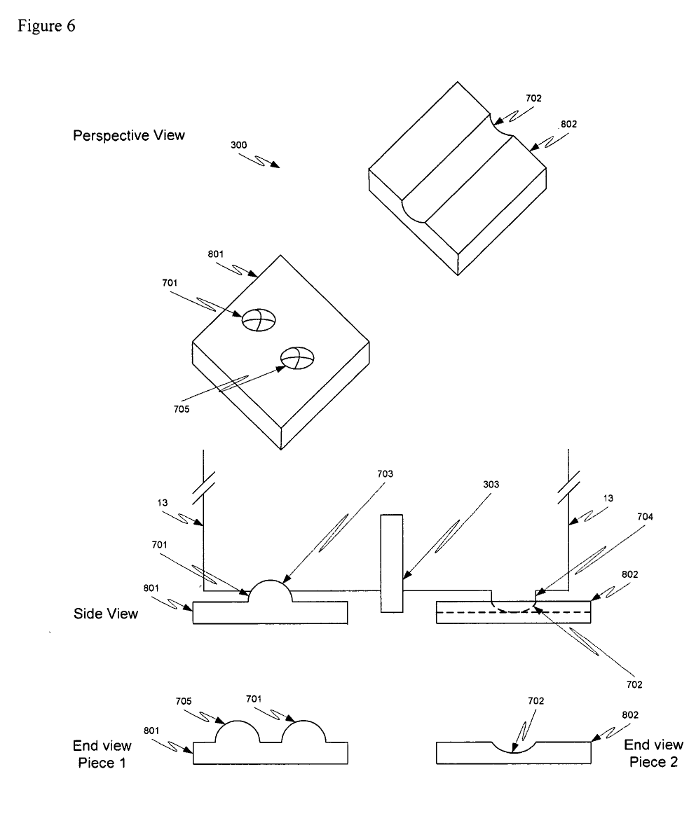

Referring now to FIG. 6, an adaptive mount similar to that of FIG. 5 is presented as a perspective view, as a side view, and as an end view. In this embodiment, the registration pieces 701, 705 have a curved upper surface that interfaces with a portion of a sample module. Alternatively, the registration pieces 701, 705 interface with a portion of a sample probe. As the sample module 13 interfaces with the mount 801 through the two registration pieces 701, 705 and the registration pieces of the module, such as registration piece 703, the sample module is orientated in terms of rotation and provides a pivot point for z-axis alignment. Restated, the two registration pieces 701, 705 cooperatively limit rotation of the sample module 13. Further, the two registration pieces 701, 705 provide a pivot point and a first hard stop for the z-axis alignment. Referring now to the side view of FIG. 6, it is observed that the sample module 13 is allowed to pivot about the y-axis, hinge upwards and downwards, through the interaction of alignment piece 703 or the sample module 13, or alternatively the sample probe, with alignment piece 701 of the alignment piece 801. This is possible due to the curvature of the alignment pieces 701 and 703 and the gap between the sample module 13 and the mount alignment piece 801. The pivot allows the right side of the sample probe to move up and down in the z-axis and to slide along the x-axis in the trough 702 as the tissue under alignment piece 802 moves up or down along the z-axis relative to the tissue under alignment piece 801. Similarly, as the alignment piece 801 torques or becomes non-planar with alignment piece 802 due to tissue changes, the sample module 13 rotates about the x-axis through the interaction of alignment pieces 701 and 703 and the curved nature of alignment pieces 702 and 704. Optionally, magnetic forces draw alignment piece 704, such as a magnetizable ball bearing, toward alignment piece 802. The small surface area contact of the alignment piece 704 against the alignment piece 702 reduces total resistance to movement along the x/y-plane. Optionally, one or more of the registration pieces, such as 701, 705, are offset from the alignment piece 801 by a distance, through such means as a post.

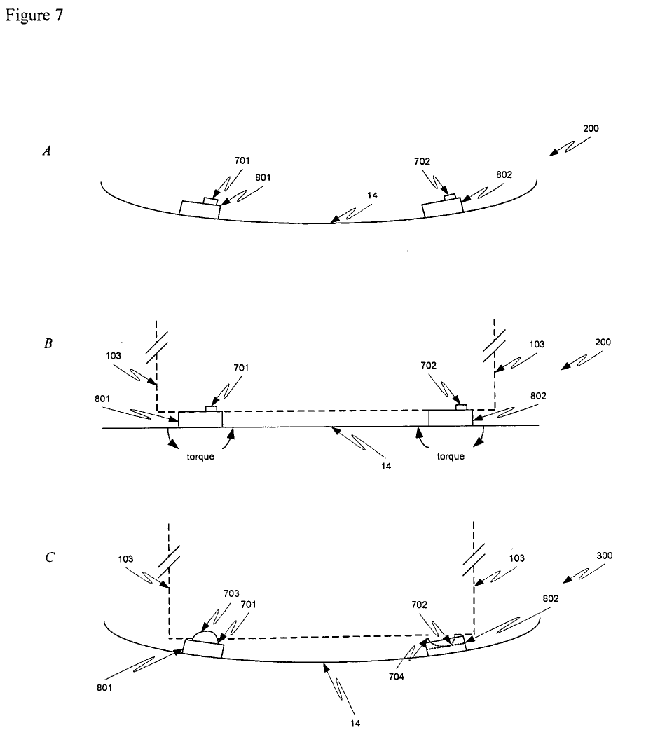

Referring now to FIG. 7, minimization of tissue strain due to torque is taught. Referring now to FIG. 7A, a first and second alignment piece 801, 802 of a two-piece guide 200 are positioned on a tissue sample 14. Each alignment piece has a corresponding registration piece 701, 702. The tissue is curved. Referring now to FIG. 7B, the elements of FIG. 7A are presented at another point in time when a sample module 13 and/or a sample probe is brought into contact with the registration pieces 701, 702. For the sample module 13 to intersect with the registration pieces 701, 702, the tissue 14 deforms through an applied torque. The alignment pieces are pushed to match the shape of the sample module, in this case the alignment pieces are forced into a coplanar arrangement. The resulting torque on the skin applies stress resulting in a strain in the tissue about the sample site, which degrades the noninvasive optical signal.

Referring now to FIG. 7C, an adaptive mount 300 is presented that minimizes torque on the tissue 14 when the sample module 13 is positioned relative to the tissue 14. The mount contains a feature that adapts alignment of the sample module to the tissue shape. In this case

, a rounded surface is used as an alignment piece 701. The interface 703 of the sample module 13 couples to the registration piece 701. The interface allows the sample module to rotate relative to the tissue. The second registration pair 702, 704 allows the sample module to slide along the second alignment piece relative to the tissue. The net result is that the sample module adapts to the shape of the tissue using an adaptive mount. This contrasts with the tissue adapting to the sample module when using a guide.

In FIG. 7C a rounded surface is used as an example of an adaptive mount mechanism. If the surface is round about the y-axis, then the sample probe pivots and allows adaptation along the x-axis. Rounding about any axis is possible. Rounding along a single axis results in one degree of freedom of adaptation. Alternative mechanisms provide similar adaptation, such as a hinge, a wing, or a flexible member. Alternatively, adaptation is allowed through two or more degrees of freedom. For example, the registration piece 701 is a ball bearing. This allows rotation about the x- and y-axes. Similar mechanisms exist, such as a ball joint or a ball socket. In still yet another embodiment of the invention, one or more registration pieces is offset from the alignment pieces through offset means, such as a post.

FIG. 6 is illustrative of a mount 300 interfacing a sample probe or sample module 13 to tissue 14. Additional embodiments include differently shaped alignment pieces and/or differently shaped registration pieces. Embodiments of alignment pieces include a variety of geometric shapes. In addition, examples of one or more possible registration pieces include:
a ball bearing;
a kinematic mount;
a hinge;
a slide;
an extrusion;
an indentation; and
a mechanical stop.

In addition, an alignment piece is optionally shaped to act as a registration piece on the surface opposite the mounting surface replaceably attached to the tissue. Conversely, a registration piece is optionally shaped to interface with the tissue on the side opposite that interfacing to the sample module or movable sample probe. Further, the registration pieces optionally work cooperatively with their corresponding alignment pieces on the sample probe, such that combined they limit one or more of the rotation and x-, y-, and z-position of the sample probe relative to the mount. For example, combined the lock and key mechanism allow for control of all or a fraction of rotation, and x-, y-, and z-position of the sample probe movement relative to the tissue. Still further, additional permutations and combination means for registering the sample probe relative to the first alignment piece are possible.

Movable Sample Probe

The sample probe is optionally controlled by the mount along approximately the z-axis. The invention optionally guides a noninvasive analyzer sample probe that applies a controlled displacement of the sample probe relative to a sample and/or a controlled movement of the sample probe along the z-axis. The z-axis control of the displaced sample probe element of the sample module provides for collection of noninvasive spectra with a given displacement of tissue, incidental contact with tissue, and/or no contact between the sample probe and the tissue sample. Preferably, the tip of the sample module is placed within about one millimeter of the nominal surface of the sample site and more preferably the sample module is place to within about two tenths of a millimeter of the nominal surface of the sample site.

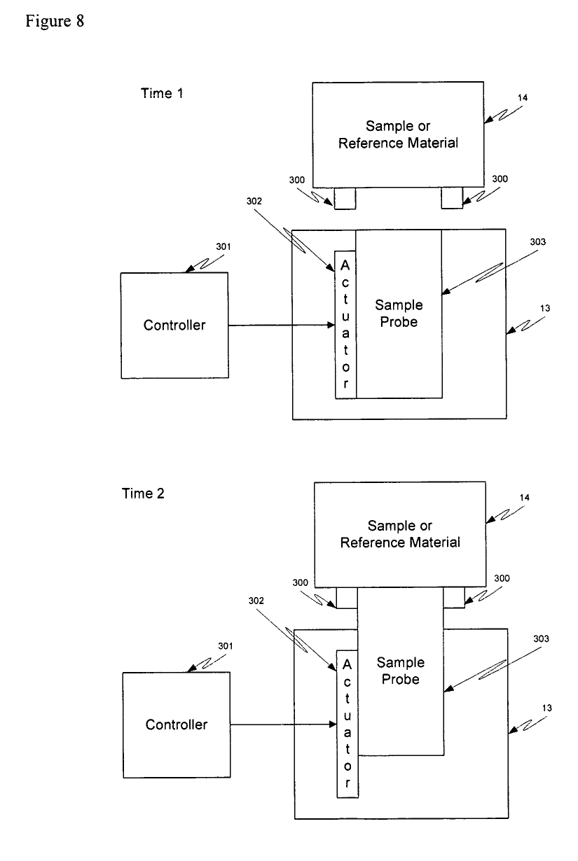

Referring now to FIG. 8, a schematic presentation of optional sample probe movement relative to a sample 14 is presented. In this embodiment, The sample module 13 includes a sample probe 303. A controller 301 controls an actuator 302 that moves the sample probe 303. Signal processing means result in a control signal that is transferred from the controller 301 to the sample probe 303 typically through an actuator 302. The communicated control signal is used to control the z-axis movement of at least part of the sample module 13 relative to the tissue sample 14 or reference material. The part of the sample module 13 movable along the z-axis is referred to as the sample probe or sampling probe 303. In one case, the controller sends the control signal from the algorithm to the sample module actuator, preferably via a communication bund1e 12. In a second case, the controller 301 receives input from the sample probe or other sensor and uses the input to move the actuator 302. Thus, in various embodiments, the controller is in different locations within the analyzer, such as in the sample module 13 or in the base module 11. In these cases, the actuator 302 subsequently moves the sample probe 303 relative to the tissue sample site 14. In a third case, no controller or actuator is used and the sample probe moves in response to gravity. The sample probe 303 is typically controlled along the z-axis from a position of no contact, to a position of tissue sample contact, and optionally to a position of tissue sample displacement.

The sample probe 303 is presented in FIG. 8 at a first and second instant of time with the first time presenting the sample probe when it is not in contact with the sample site. The second time presents the sample probe with minimal contact and/or displacement of the sample tissue. The sample probe is, optionally, moved toward the sample, away from the sample, or remains static as a function of time. The replaceably attached mount 300 is attached to the sample and/or reference. Input to the controller 301 includes a predetermined profile, an interpretation of spectral data collected from the sample probe 303, or input from a sensor, such as a pressure sensor, an optical sensor, a distance sensor, a position sensor, a tilt sensor, or a thermal sensor. In yet another embodiment of the invention a gimbal ring, which is a device consisting of two rings mounted on axes at right angles to each other so that an object, such as a probe, remains suspended in a horizontal plane between them regardless of any motion of its support is used. Additional embodiments of a movable z-axis probe are described in U.S. provision patent application no. 60/566,568, which is incorporated herein in its entirety by this reference thereto.

In additional embodiments, the sample probe is movable along any of the x-, y-, and z-axis and/or in terms of tilt or rotation prior to interfacing with the sample site where the distance between the sample site and the tip of the sample probe and/or the tilt of the sample probe relative to the sample site is determined and controlled using one or more control sensors. The control sensors include one or more of capacitive, magnetic, optical, current, inductive, ultrasonic, resistive and electrical contact based sensors as described in U.S. provisional patent application no 60/761,486 filed Jan. 23, 2006 (attorney docket no. SENS0065PR), which is incorporated herein in its entirety by this reference thereto.

Those skilled in the art will recognize that the present invention may be manifested in a variety of forms other than the specific embodiments described and contemplated herein. Departures in form and detail may be made without departing from the spirit and scope of the present invention. Accordingly, the invention should only be limited by the Claims included below.

1. An adaptive mount for coupling a sample probe to a sample site of a tissue, comprising:
a first alignment piece; and
a second alignment piece;
wherein both said first alignment piece and said second alignment piece replaceably abut said tissue,
wherein said first alignment piece and said second alignment piece cooperate to position said sample probe relative to said sample site during use; and
wherein said adaptive mount adapts said position of said sample probe relative to said tissue as either distance between, or orientation of, said first alignment piece and said second alignment piece change due to state change of said tissue.

2. The mount of claim 1, wherein said position floats in terms of at least one of an x-axis and a y-axis relative to a fixed x,y-sample site position; and wherein said x-axis is along the length of a body part and said y-axis is across said body part.

3. The mount of claim 1, further comprising:
a first registration piece connected to said first alignment piece; and
a second registration piece connected to said second alignment piece,
wherein said first registration piece and said second registration piece cooperatively orientate said sample probe relative to said tissue in at least two of:
a x-axis position;
a y-axis position; and
a z-axis position.

4. The mount of claim 3, wherein either said first registration piece or said second registration piece comprise any of:
a ball bearing;
a kinematic mount;
a hinge;
a slide;
an extrusion from said first and second alignment piece, respectively;
an indentation into said first and second alignment piece, respectively; and
a mechanical stop.

5. An apparatus for noninvasively measuring an analyte property at a sample site of tissue, comprising:
a base module;
a sample module coupled to said base module, said sample module having a tip; and
an adaptive mount replaceably coupled to said sample module,
wherein said adaptive mount positions with freedom of movement along at least one of an x-axis and a y-axis said sample probe tip relative to a sample site of said tissue as said tissue changes state in terms of any of:
elongation;
expansion;
contraction; and
twist orientation,
wherein said x-axis is along the length of a body part and said y-axis is across said body part.

6. The apparatus of claim 5, further comprising:
a controller controlling, via an actuator, movement of said sample probe tip along an axis about normal to a surface defined by said x-axis and y-axis.

7. The apparatus of claim 6, where said controller operates using signal from a sensor, wherein said sensor comprises any of:
a pressure sensor;
an optical sensor;
a distance sensor;
a position sensor;
a tilt sensor;
a thermal sensor; and
a contact sensor.

8. The apparatus of claim 7, wherein said controller is used in positioning said sample probe tip within one millimeter of said sample site.

9. The apparatus of claim 8, wherein said controller controls tilt of said sample probe relative to said tissue.

10. The apparatus of claim 5, wherein said sample module and said base module are communicatively connected using any of:
wireless communication; and
a communication bund1e.

11. The apparatus of claim 5, wherein said base module comprises a photo-diode array detector and wherein said sample module comprises a source.

12. The apparatus of claim 5, wherein said sample module and said base module are combined into a handheld analyzer.

13. A method for noninvasive analysis of a tissue sample glucose concentration, comprising the step of:
moving a sample probe tip into proximate contact with a tissue sample site, with freedom of movement along at least one of an x-axis and a y-axis relative to said sample site as tissue at said sample site changes state in terms of any of:
elongation;
expansion;
contraction; and
twist orientation;
wherein said x-axis is along the length of a body part and said y- axis is across said body part;
wherein said means for moving are operatively coupled to an analyzer;
acquiring with said analyzer a noninvasive near-infrared spectrum of said tissue sample; and
determining glucose concentration representative of said tissue sample by applying a multivariate analysis to said noninvasive spectrum.

14. An apparatus for to coupling a sample probe to a sample site of tissue, comprising:
a mount in proximate contact with said tissue during use;
wherein said mount is used in conjunction with a noninvasive analyzer having a sample probe;and
wherein during use said mount adapts position of said sample probe relative to said tissue as said tissue changes state in terms of any of:
response to a stress;
an elongation;
a contraction; and
a twist.

15. The apparatus of claim 14, wherein said position of said sample probe comprises an x,y-location of said sample probe; and wherein said x-axis is along the length of a body part and said y-axis is across said body part.

16. An adaptive mount used to couple a sample probe to a sample site of a tissue, comprising:
a first alignment piece having a contact surface, at least a portion of said contact surface being in contact with a surface proximate said tissue during use; and
a second alignment piece having a tissue side surface, at least a portion of said tissue side surface being in contact with said surface proximate said tissue during use;
wherein said first alignment piece and said second alignment piece move with said tissue as said tissue changes state in terms of any of expansion, contraction, and twist;
wherein said first alignment piece and said second alignment piece cooperatively position said sample probe of an analyzer to said sample site; and
wherein said sample site varies with said change in tissue state.

17. The adaptive mount of claim 16, wherein said change in tissue state comprises any of:
an elongation;
a contraction; and
a twist.

18. The adaptive mount of claim 16, wherein distance between said first alignment piece and said second alignment piece varies with tissue state and said distance is nominally unchanged during the step of coupling of said mount to said sample probe.

19. The mount of claim 16, wherein said analyzer comprises a noninvasive near-infrared based glucose concentration analyzer.
